(19) United States
(12) Patent Application Publication
(10) Pub. No.: US 2021/0361178 A1
(43) Pub. Date: Nov. 25, 2021

(54) BLOOD PRESSURE LEVEL CHANGE DETECTION APPARATUS, AND METHOD FOR DETECTING CHANGE IN BLOOD PRESSURE LEVEL

(71) Applicant: OMRON HEALTHCARE CO., LTD., Muko-shi (JP)

(72) Inventors: Tatsunori ITO, Kyoto (JP); Shingo YAMASHITA, Kyoto (JP); Mitsuo KUWABARA, Kyoto (JP); Yuki OTA, Kyoto (JP); Ayako KOKUBO, Kyoto (JP)

(73) Assignee: OMRON HEALTHCARE CO., LTD., Muko-shi (JP)

(21) Appl. No.: 17/398,431

(22) Filed: Aug. 10, 2021

Related U.S. Application Data
(63) Continuation of application No. PCT/JP2020/004827, filed on Feb. 7, 2020.

(30) Foreign Application Priority Data
Feb. 18, 2019 (JP) 2019-026760

(51) Int. Cl. A61B 5/021; A61B 5/00

(52) U.S. Cl. CPC A61B 5/02125; A61B 5/6824; A61B 2560/02; A61B 5/0004; A61B 2560/04; A61B 5/742

(57) ABSTRACT

A blood pressure level change detection apparatus includes a change point detection unit and a level change/return determination unit. The change point detection unit detects a first change point as a change point in time-series data of blood pressure. A level change/return determination unit acquires a first average blood pressure level and a second average blood pressure level for time-series data of blood pressure. When a difference between the first average blood pressure level and the second average blood pressure level is greater than or equal to a predetermined level threshold value, the level change/return determination unit determines that a blood pressure level change has occurred at the first change point.

### CROSS-REFERENCE TO RELATED APPLICATIONS

The present application is a continuation application of International Application No. PCT/JP2020/004827, filed Feb. 7, 2020, which claims priority to Japanese Patent Application No. 2019-026760, filed Feb. 18, 2019. The contents of these applications are incorporated herein by reference in their entirety.

### BACKGROUND

### Field

The present invention relates to a blood pressure level change detection apparatus, and a blood pressure level change detection method.

### Description of the Related Art

Conventionally, blood pressure is continuously measured for each beat. For example, Japanese Unexamined Patent Application Publication No. 2018-42606 discloses that an artery near a wrist of a subject is pressed to continuously measure blood pressure for each beat.

### SUMMARY OF THE INVENTION

According to a one aspect of the present invention, a blood pressure level change detection apparatus for detecting a blood pressure level change in time-series data of blood pressure includes a change point detection unit and a level change determination unit. The change point detection unit detects a first change point as a change point representing a time at which a blood pressure value has changed beyond a predetermined change rate in the time-series data of blood pressure. The level change determination unit acquires a first average blood pressure level by averaging blood pressure values in a period of a continuous predetermined length immediately before the first change point and acquires a second average blood pressure level by averaging blood pressure values in a period of a continuous predetermined length immediately after the first change point for the time-series data of blood pressure, and determines that a blood pressure level change has occurred at the first change point when a difference between the first average blood pressure level and the second average blood pressure level is equal to or greater than a predetermined level threshold value.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings.

FIG. 1 is a diagram illustrating a schematic configuration of a blood pressure level change detection system according to an embodiment.

FIG. 2 is a diagram illustrating an attachment state of a sphygmomanometer included in the blood pressure level change detection system.

FIG. 3 is a cross-sectional view illustrating an attachment state of the sphygmomanometer included in the blood pressure level change detection system.

FIG. 4 is a diagram illustrating a schematic configuration of the sphygmomanometer included in the blood pressure level change detection system.

FIG. 5 is a diagram illustrating a schematic configuration of a blood pressure level change detection apparatus included in the blood pressure level change detection system.

FIG. 6 is a flowchart for explaining an operation of determining presence or absence of a blood pressure level change.

FIG. 7 is a diagram for explaining an operation of detecting a change point in time-series data of a maximum blood pressure value.

FIG. 8 is a diagram for explaining an operation of setting a section based on the change point in the time-series data of the maximum blood pressure value.

FIG. 9 is a diagram illustrating a schematic configuration of a blood pressure level change detection apparatus according to another embodiment.

FIG. 10 is a diagram for explaining an operation of determining validity of a detected change point.

FIG. 11 is a flowchart illustrating an operation of determining the validity of the detected change point.

### DESCRIPTION OF THE EMBODIMENTS

The embodiments will now be described with reference to the accompanying drawings, wherein like reference numerals designate corresponding or identical elements throughout the various drawings.

Hereinafter, embodiments of the present invention will be described in detail with reference to the drawings.

### First Embodiment

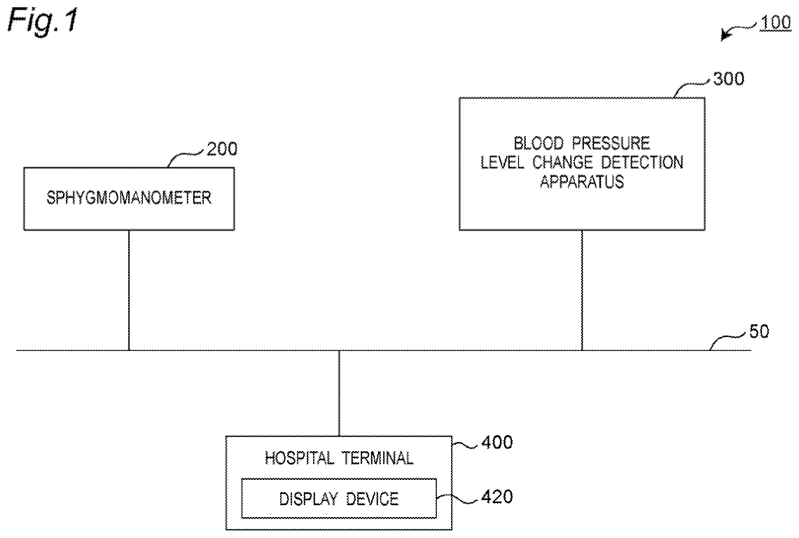

FIG. 1 illustrates a schematic configuration of a blood pressure level change detection system 100 according to a first embodiment. The blood pressure level change detection system 100 includes a tonometry-type sphygmomanometer 200, a blood pressure level change detection apparatus 300, and at least one or more hospital terminals 400. As illustrated in FIG. 1, the sphygmomanometer 200, the blood pressure level change detection apparatus 300, and the hospital terminal 400 are communicably connected to each other via a communication network 50. Here, the communication network 50 may be wireless or wired.

### (Schematic Configuration of Sphygmomanometer 200)

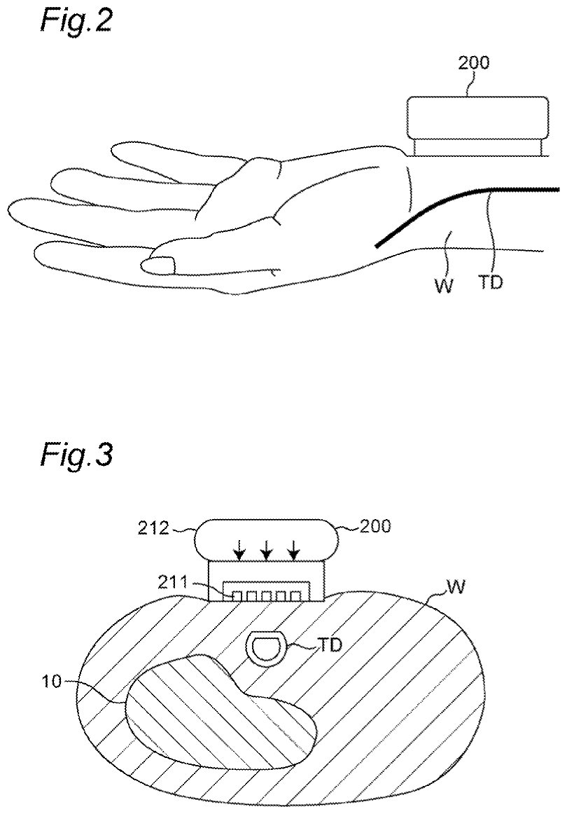

The sphygmomanometer 200 illustrated in FIG. includes, for example, a tonometry-type sphygmomanometer as disclosed in JP 2018-42606 A. FIG. 2 illustrates a state in which the sphygmomanometer 200 is attached to a wrist w of a subject. FIG. 3 is a cross-sectional view illustrating a state in which the sphygmomanometer 200 attached to the wrist w of the subject performs blood pressure measurement. The sphygmomanometer 200 illustrated in FIGS. 2 and 3 continuously measures a pressure pulse wave of a radial artery TD traveling along a radius 10 for each beat.

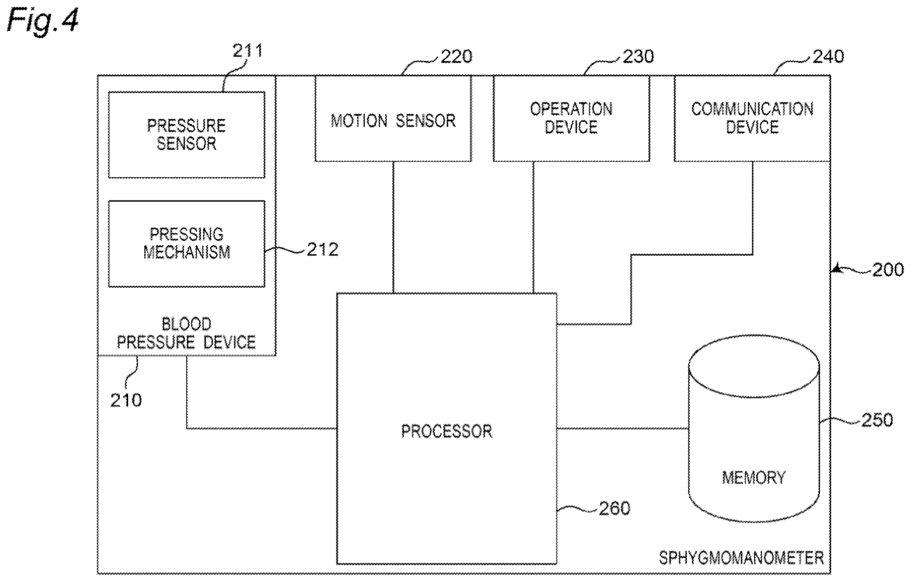

FIG. 4 illustrates a schematic configuration of the sphygmomanometer 200. As illustrated in FIG. 4, the sphygmomanometer 200 includes a blood pressure device 210, a motion sensor 220, an operation device 230, a communication device 240, a memory 250, and a processor 260. In addition, the blood pressure device 210 includes a pressure sensor 211 and a pressing mechanism 212.

As illustrated in FIG. 3, the pressing mechanism 212 applies a pressing force to a measurement target site. When the pressing mechanism 212 applies a pressing force to the measurement target site, the pressure sensor 211 continuously detects the pressure pulse wave of the radial artery TD for each beat by tonometry. Tonometry is a method in which a blood vessel is flattened using the pressing mechanism 212, and the pressure sensor 211 measures a pressure pulse wave to determine blood pressure. When the blood vessel is regarded as a circular tube having a uniform thickness, a relational expression between an internal pressure (blood pressure) of the blood vessel and an external pressure (pressure of the pressure pulse wave) of the blood vessel can be derived according to Laplace's law in consideration of the blood vessel wall regardless of a flow of blood in the blood vessel and presence or absence of pulsation. Under a condition that the blood vessel is flattened on a pressed surface in this relational expression, the pressure of the pressure pulse wave and the blood pressure can be approximated to be equal by approximating radii of an outer wall and an inner wall of the blood vessel. Therefore, the pressure of the pressure pulse wave has the same value as the blood pressure. As a result, the sphygmomanometer 200 measures the blood pressure value at the measurement target site for each heartbeat. Then, the sphygmomanometer 200 generates time-series data of blood pressure in which the measurement time (time) is associated with the blood pressure, and outputs the time-series data to another device (for example, the blood pressure level change detection apparatus 300).

In FIG. 4, the motion sensor 220 is a sensor that detects a motion of the sphygmomanometer 200. The motion sensor 220 includes, for example, an acceleration sensor and/or an angular velocity sensor. The operating device 230 receives an instruction (input) from a user. The operating device 230 includes, for example, a plurality of buttons. The communication device 240 transmits and receives various types of data. In the example of FIG. 1, the communication device 240 is connected to the communication network 50. The memory 250 stores various types of data. For example, the memory 250 can store a measurement value measured by the blood pressure device 210 (time-series data of blood pressure described above), a measurement result of the motion sensor 220, and the like. The memory 250 includes a random access memory (RAM), a read only memory (ROM), and the like. For example, various programs are changeably stored in the memory 250.

In this example, the processor 260 includes a central processing unit (CPU). For example, the processor 260 reads each program and each data stored in the memory 250. In addition, the processor 260 controls each of the units 210, 220, 230, 240, and 250 according to the read program to execute a predetermined operation (function). In addition, the processor 260 performs predetermined calculation, analysis, processing, and the like in the processor 260 according to the read program. Note that some or all of the functions executed by the processor 260 may be configured as hardware by one or more integrated circuits or the like.

### (Schematic Configuration of Blood Pressure Level Change Detection Apparatus 300)

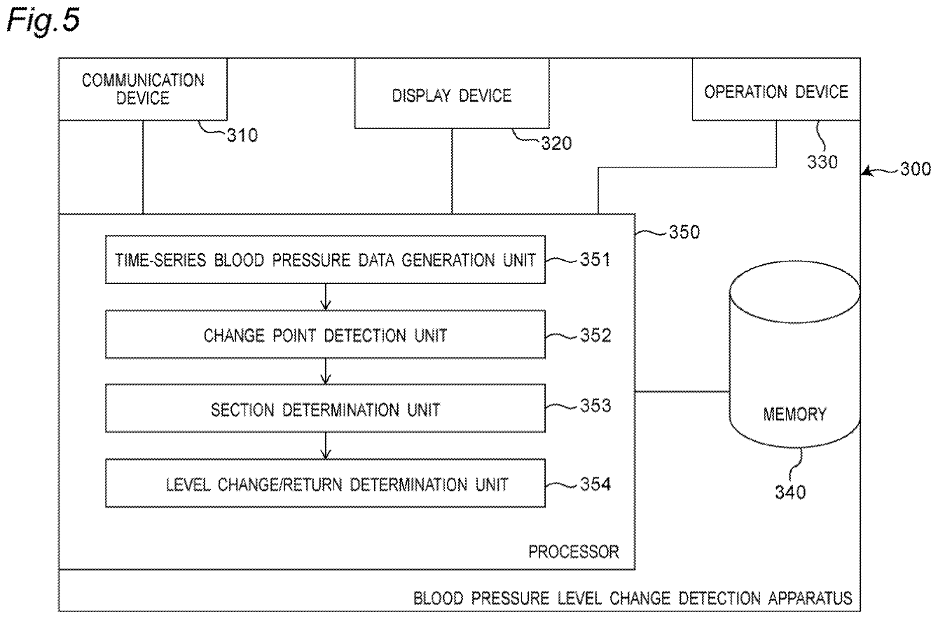

The blood pressure level change detection apparatus 300 according to the present embodiment detects a blood pressure level change in time-series data of blood pressure. Here, in the present embodiment, the time-series data of blood pressure is obtained from the measurement result of the sphygmomanometer 200. FIG. 5 illustrates a schematic configuration of the blood pressure level change detection apparatus 300. As illustrated in FIG. 5, the blood pressure level change detection apparatus 300 includes a communication device 310, a display device 320, an operation device 330, a memory 340, and a processor 350.

In FIG. 5, the communication device 310 transmits and receives various types of data. In the example of FIG. 1, the communication device 310 is connected to the communication network 50. The communication device 310 receives, for example, the time-series data of blood pressure and a detection result of the motion sensor 220 transmitted from the sphygmomanometer 200. Furthermore, the communication device 310 can also transmit various output data generated by the processor 350 in the blood pressure level change detection apparatus 300 to the hospital terminal 400 or the like.

The display device 320 has a display screen that displays various images. The display device 320 can display, in a visually recognizable manner, results of various types of analysis and the like in the processor 350. Furthermore, the display device 320 can also display predetermined information in a visually recognizable manner according to a desire from the user via the operation device 330. For example, the display device 320 may display information (data) stored in the memory 340 in a visually recognizable manner. For example, a liquid crystal monitor or the like can be adopted as the display device 320.

The operating device 330 receives a predetermined operation (instruction) from the user. For example, the operation device 330 includes a mouse, a keyboard, and the like. Here, in a case where a touch panel monitor is employed as the display device 320, the display device 320 has not only a display function but also a function as the operation device 330.

The memory 340 stores various types of data. For example, the memory 340 can store a measurement value measured by the blood pressure device 210 (time-series data of blood pressure described above), a measurement result of the motion sensor 220, and the like. The memory 340 can also store various output data generated by the processor 350. The memory 340 includes a RAM, a ROM, and the like. For example, various programs are changeably stored in the memory 340.

The processor 350 includes a CPU in this example. For example, the processor 350 reads each program and each data stored in the memory 340. In addition, the processor 350 controls each of the units 310, 320, 330, and 340 according to the read program to execute a predetermined operation (function). In addition, the processor 350 performs predetermined calculation, analysis, processing, and the like in the processor 350 according to the read program. Note that some or all of the functions executed by the processor 350 may be configured as hardware by one or a plurality of integrated circuits or the like.

As illustrated in FIG. 5, the processor 350 according to the present embodiment includes, as functional blocks, a time-series blood pressure data generation unit 351, a change point detection unit 352, a section determination unit 353, and a level change determination/level return determination unit 354. Note that the operation of each of the blocks 351, 352, 353, and 354 will be described in detail in the description of the operation to be described later.

### (Schematic Configuration of Hospital Terminal 400)

The hospital terminal 400 illustrated in FIG. 1 is configured by a general personal computer in this example. Note that, as described above, a plurality of the hospital terminals 400 may be disposed in the system configuration illustrated in FIG. 1. Here, the hospital terminal 400 may be a portable terminal such as a tablet instead of the personal computer.

A display device 420 included in the hospital terminal 400 has a display screen that displays various images. For example, the display device 420 displays, in a visually recognizable manner, an image based on various output data received from the blood pressure level change detection apparatus 300. Furthermore, the display device 420 can also display predetermined information in a visually recognizable manner according to an operation by the user. For example, a liquid crystal monitor or the like can be adopted as the display device 420.

### (Operation of Blood Pressure Level Change Detection System 100)

The blood pressure level change detection method performed by the blood pressure level change detection system 100 is a method for detecting a blood pressure level change in time-series data of blood pressure. As described above, the time-series data of blood pressure is obtained from the measurement result of the sphygmomanometer 200.

### (1) Operation of Sphygmomanometer 200

Measurement is performed in the sphygmomanometer 200. Note that the measurement includes measurement of a blood pressure value for each beat by the blood pressure device 210 and detection measurement of movement of the sphygmomanometer 200 by the motion sensor 220. Note that the blood pressure data for each beat is associated with the measurement time, and similarly, each motion data is also associated with the measurement time.

As described above, for example, the sphygmomanometer 200 is attached to the wrist w of the subject in order to continuously measure the pressure pulse wave (blood pressure) of the radial artery TD for each beat (see FIGS. 2 and 3). Then, at the time of the blood pressure measurement, the pressing mechanism 212 of the blood pressure device 210 applies a predetermined pressing force to the wrist w. Then, while the pressing force is applied, the pressure sensor 211 of the blood pressure device 210 detects the blood pressure of the radial artery TD for each beat. Note that the detection result of the motion sensor 220 is stored in time series in the memory 250 of the sphygmomanometer 200, for example. Similarly, the measurement results of the pressure sensor 211 are stored in time series in the memory 250.

Next, the communication device 240 f the sphygmomanometer 200 transmits the measurement data to the blood pressure level change detection apparatus 300 in this example. Here, the measurement data includes the detection result of the motion sensor 220 and the measurement result of the pressure sensor 211. The communication device 310 of the blood pressure level change detection apparatus 300 receives the transmitted measurement data. The memory 340 of the blood pressure level change detection apparatus 300 stores the measurement data received by the communication device 310. As described above, the blood pressure data for each beat is associated with the measurement time, and similarly, each motion data is also associated with the measurement time. In addition, the memory 340 stores measurement results of the pressure sensor 211 in time series. Furthermore, the memory 340 stores detection results of the motion sensor 220 in time series. The sphygmomanometer 200 may once transmit the measurement data to any one of the hospital terminals 400, and the hospital terminal 400 may transmit the measurement data to the blood pressure level change detection apparatus 300.

### (2) Operation of Blood Pressure Level Change Detection Apparatus 300

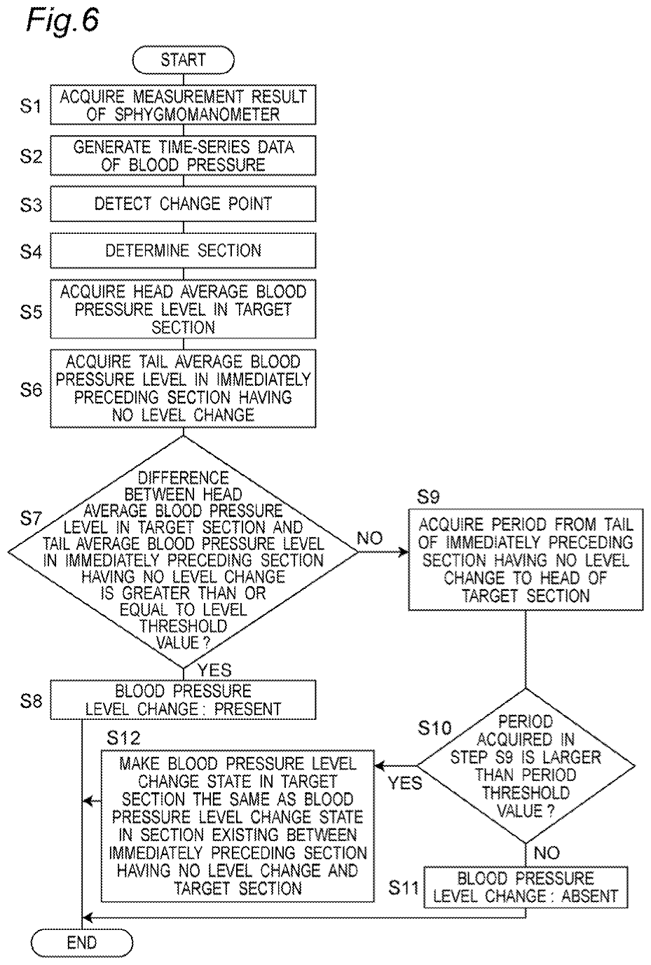

When the blood pressure measurement is continuously performed for a long time (for example, overnight) using the sphygmomanometer 200, body motion of the subject may occur during the measurement. Then, a height or the like of the sphygmomanometer 200 with respect to the heart of the subject changes with the body motion as a trigger, and a blood pressure level change in the time-series data of blood pressure (a phenomenon in which the blood pressure value steeply changes from a certain level to another level) may occur. Next, the operation of the blood pressure level change detection in the blood pressure level change detection apparatus 300 will be specifically described with reference to the flowchart illustrated in FIG. 6.

The time-series data of blood pressure includes time-series data of a maximum blood pressure value (alternatively, systolic blood pressure) and time-series data of a minimum blood pressure value (alternatively, diastolic blood pressure). As the time-series data of blood pressure, the time-series data of the minimum blood pressure value may be adopted. However, in the following description, as an example, the time-series data of blood pressure is the time-series data of the maximum blood pressure value.

As described above, the blood pressure level change detection apparatus 300 receives the measurement data, and the memory 340 of the blood pressure level change detection apparatus 300 stores the measurement data. Here, the measurement data includes data measured by the blood pressure device 210 of the sphygmomanometer 200 (blood pressure value for each beat). As described above, the blood pressure value for each beat is associated with the measurement time point for the blood pressure value of one beat.

First, in step S1 of FIG. 6, the time-series blood pressure data generation unit 351 of the processor 350 reads each of the blood pressure values for each beat from the memory 340, and acquires the maximum blood pressure value from the read blood pressure value for each beat.

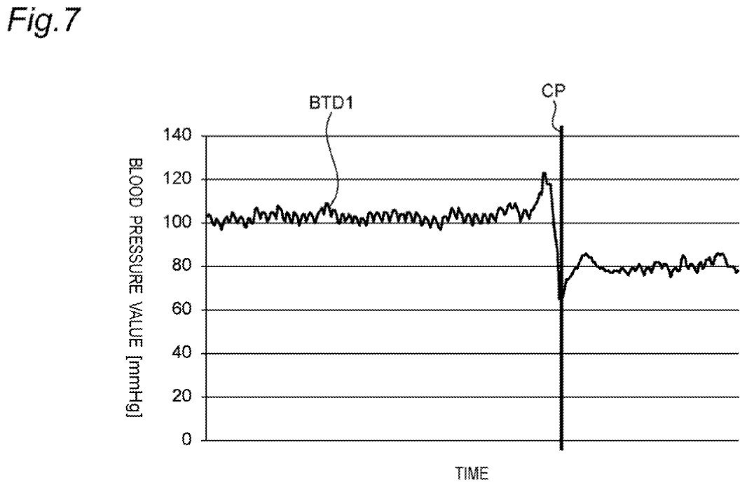

Next, the time-series blood pressure data generation unit 351 generates time-series data BTD1 of blood pressure (in the present embodiment, the maximum blood pressure) using each of the maximum blood pressure values of one beat acquired in step S1 (step S2). The time-series data BTD1 of the maximum blood pressure is generated by arranging each of the maximum blood pressure values of one beat in time series. FIG. 7 illustrates an example of the generated time-series data BTD1 of the maximum blood pressure. Here, a vertical axis in FIG. 7 represents the blood pressure value (mmHg), and a horizontal axis in FIG. 7 represents time.

Next, the change point detection unit 352 of the processor 350 detects a change point CP (see FIG. 7) in the time-series data BTD1 of the maximum blood pressure (step S3). Here, in the present embodiment, the change point represents a time when a tendency of the maximum blood pressure value changes steeply. Specifically, the change point represents a time at which the blood pressure value (in the present embodiment, maximum blood pressure value) for each beat changes beyond a predetermined change rate. For example, the change point is detected using a generally known change finder method, a method using a likelihood ratio test, a method using an auto-regressive (AR) model, or a method disclosed in JP 2018-147442 A.

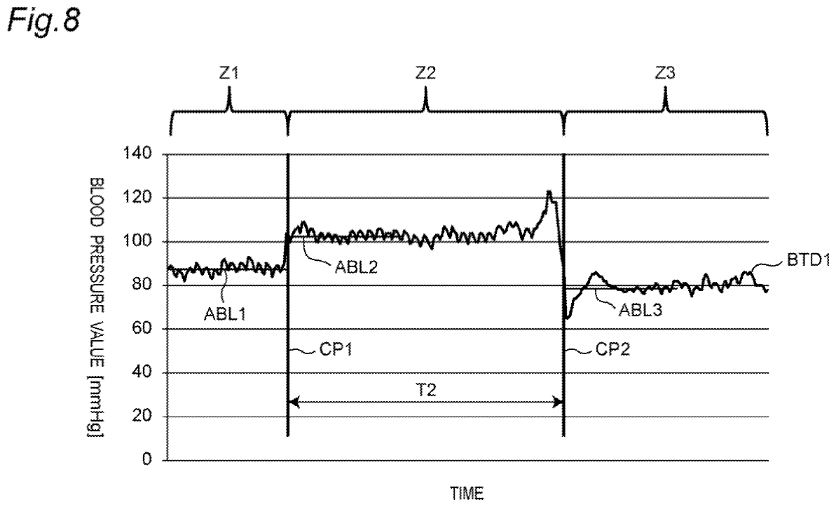

In the following description, it is assumed that the change point detection unit 352 detects at least a first change point CP1 and a second change point CP2 as the change point CP in step S3 as illustrated in FIG. 8. In FIG. 8, a vertical axis represents the blood pressure value (mmHg), and a horizontal axis represents time. Here, the detection point CP that first appears in time-series after the start of measurement with the sphygmomanometer 200 is the first detection point CP1. The detection point CP after the first detection point CP1 is the second detection point CP2. In other words, the detection point CP that appears next to the first detection point CP1 in time-series is the second detection point CP2.

Next, the section determination unit 353 of the processor 350 determines a plurality of consecutive sections in the time-series data BTD1 of the maximum blood pressure based on the change point CP, and divides the time-series data BTD1 of the maximum blood pressure into the sections (step S4). As described above, when the two detection points CP1 and CP2 are detected, the section determination unit 353 divides the time-series data BTDI of the maximum blood pressure into continuous first section Z1, second section Z2, and third section Z3 by the first change point CP1 and the second change point CP2 (see FIG. 8). That is, the section determination unit 353 determines the first section Z1, the second section Z2, and the third section Z3 with the change points CP1 and CP2 as boundaries. Then, the section determination unit 353 divides the time-series data BTD1 of the maximum blood pressure into the sections Z1, Z2, and Z3. Therefore, the first change point CP1 exists between the first section Z1 and the second section Z2 (boundary), and the second change point CP2 exists between the second section Z2 and the third section Z3 (boundary).

Here, in the example of FIG. 8, the first section Z1 is a period of the time-series data BTD1 of the maximum blood pressure from a start time point of blood pressure measurement by the sphygmomanometer 200 to the first change point CP1 detected first after the start of the measurement. The second section Z2 is a period of the time-series data BTD1 of the maximum blood pressure from the time point of the first change point CP1 to the second change point CP2. The third section Z3 is a period of the time-series data BTD1 of the maximum blood pressure from the time point of the second change point CP2 to the third change point (not illustrated) (or until the end of the blood pressure measurement if no change point is detected thereafter). As described above, the first section Z1 is a period from immediately after the start of the measurement to the detection of the initial first change point CP1. Therefore, the first section Z1 is defined as a section in which there is no blood pressure level change, and is used as a reference in the subsequent determination of the blood pressure level change.

In the above description, the first section Z1 is adopted as the “reference”. That is, it is determined whether the blood pressure level changes in the subsequent sections Z2 and Z3 with respect to the first section Z1. However, the first section Z1 may not be adopted as the reference, and for example, a blood pressure value separately measured by a method resistant to disturbance may be adopted as the reference. As the blood pressure value measured by a method resistant to the disturbance, for example, a blood pressure value measured by a conventional upper-arm sphygmomanometer can be adopted.

After step S4, the level change/return determination unit 354 performs each of steps S5 to S12 after step S5 on each of the sections Z2 and Z3 after the second section Z2. Then, in each of the sections Z2 and Z3, the level change/return determination unit 354 determines presence or absence of a blood pressure level change from the immediately preceding section having no level change (steps S8 and S11).

First, the level change/return determination unit 354 acquires a head average blood pressure level in a target section (step S5). Here, the target section is a section in which presence or absence of the blood pressure level change is determined, and the target section here is the second section Z2. In addition, the head average blood pressure level in the second section Z2 is an average of the maximum blood pressure values over a period of a continuous predetermined length immediately after the first change point CP1 for the time-series data BTD1 of the maximum blood pressure. Here, the predetermined period is variably set in advance in the blood pressure level change detection apparatus 300. As an example, the predetermined length may be a length of 100 beats of blood pressure. Here, the level change/return determination unit 354 averages the maximum blood pressure values over the continuous period of the predetermined length immediately after the first change point CP1. In this example, the result of the average is expressed as a second average blood pressure level ABL2. Therefore, the level change/return determination unit 354 acquires the second average blood pressure level ABL2 as the head average blood pressure level in the target section Z2 (see FIG. 8).

Next, the level change/return determination unit 354 acquires a tail average blood pressure level in the immediately preceding section having no level change (step S6). Here, the immediately preceding section having no level change is a section before the target section, in which it has been determined that there is no blood pressure level change. Here, the immediately preceding section having no level change is a section before the target section Z2, in which it can be grasped that there is no blood pressure level change. In the example of FIG. 8, the section before the target section Z2 is only the first section Z1, and as described above, the first section Z1 is a reference section having no blood pressure level change. Therefore, in the example of FIG. 8, when the target section is the second section Z2, the immediately preceding section having no level change is the first section Z1.

The tail average blood pressure level in the first section Z1 is an average of the maximum blood pressure values over the period of the predetermined length (100 beats in this example) that is continuous immediately before the first change point CP1 for the time-series data BTD1 of the maximum blood pressure. Here, the level change/return determination unit 354 averages the maximum blood pressure values over the period of the predetermined length immediately before the first change point CP1. The result of the average is expressed as a first average blood pressure level ABL1. Therefore, the level change/return determination unit 354 acquires the first average blood pressure level ABL1 as the tail average blood pressure level in the immediately preceding section Z1 having no level change (see FIG. 8).

Next, the level change/return determination unit 354 compares a difference between the head average blood pressure level in the target section and the tail average blood pressure level in the immediately preceding section having no level change with a level threshold value (this is referred to as ABLth) (step S7). Here, a value of 5 to 50 mmHg may be adopted as the level threshold value ABLth, but the level threshold value ABLth is not limited thereto. The level threshold value ABLth is stored in advance in the memory 340 of the blood pressure level change detection apparatus 300. Therefore, the level change/return determination unit 354 reads the level threshold value ABLth from the memory 340. The level threshold value ABLth may be a changeable value or a fixed value. Furthermore, the level threshold value ABLth may be automatically calculated based on a predetermined statistical distribution or the like. Then, the calculated values may be automatically set. The matters related to the setting of the “threshold values” similarly apply to each “threshold value” which will be described below.

Here, the head average blood pressure level in the target section Z2 is the second average blood pressure level ABL2, and the tail average blood pressure level in the immediately preceding section Z1 having no level change is the first average blood pressure level ABL1. Therefore, in step S7, the level change/return determination unit 354 determines whether or not the difference between the second average blood pressure level ABL2 and the first average blood pressure level ABL1 is greater than or equal to the level threshold value ABLth.

For example, it is assumed that the level change/return determination unit 354 determines that the difference between the first average blood pressure level ABL1 and the second average blood pressure level ABL2 is equal to or larger than the level threshold value ABLth (“YES” in step S7). In this case, the level change/return determination unit 354 determines that there is a blood pressure level change in the second section Z2 (first change point CP1) (step S8). Then, the level change/return determination unit 354 records, in the memory 340, that there is a blood pressure level change with respect to all the blood pressures of one beat belonging to the second section Z2 (step S8).

On the other hand, it is assumed that the level change/return determination unit 354 determines that the difference between the second average blood pressure level ABL2 and the first average blood pressure level ABL1 is less than the level threshold value ABLth (“NO” in step S7). In this case, the level change/return determination unit 354 proceeds to step S9.

Here, in the following description, it is assumed that the level change/return determination unit 354 determines that there is a blood pressure level change in the second section Z2 (first change point CP1). Therefore, the processing of steps S9 to S12 will be described later.

Next, operations after step S5 in FIG. 6 when the target section is the third section Z3 will be described.

First, the level change/return determination unit 354 acquires a head average blood pressure level in a target section (step S5). The target section here is the third section Z3. In addition, the head average blood pressure level in the third section Z3 is an average of the maximum blood pressure values over the period of the predetermined length (100 beats in this example) which is continuous immediately after the second change point CP2 for the time-series data BTD1 of the maximum blood pressure. The level change/return determination unit 354 averages the maximum blood pressure values over the continuous period of the predetermined length immediately after the second change point CP2. The result of the average is a third average blood pressure level ABL3. Therefore, the level change/return determination unit 354 acquires the third average blood pressure level ABL3 as the head average blood pressure level in the target section Z3 (see FIG. 8).

Next, the level change/return determination unit 354 acquires a tail average blood pressure level in the immediately preceding section having no level change (step S6). Here, it is assumed that there is a blood pressure level change in the second section Z2, and as described above, the first section Z1 is the reference section. Therefore, the immediately preceding section having no level change is the first section Z1. Therefore, in step S6, the level change/return determination unit 354 acquires the first average blood pressure level ABL1 as the tail average blood pressure level in the immediately preceding section Z1 having no level change (see FIG. 8).

Next, in step S7, the level change/return determination unit 354 compares the difference between the head average blood pressure level in the target section and the tail average blood pressure level in the immediately preceding section having no level change with the level threshold value ABLth. Here, the head average blood pressure level in the target section Z3 is the third average blood pressure level ABL3, and the tail average blood pressure level in the immediately preceding section Z1 having no level change is the first average blood pressure level ABL1. Therefore, in step S7, the level change/return determination unit 354 determines whether or not the difference between the third average blood pressure level ABL3 and the first average blood pressure level ABL1 is greater than or equal to the level threshold value ABLth.

Here, it is assumed that the level change/return determination unit 354 determines that the difference between the third average blood pressure level ABL3 and the first average blood pressure level ABL1 is greater than or equal to the level threshold value ABLth (“YES” in step S7). In this case, the level change/return determination unit 354 determines that there is a blood pressure level change in the third section Z3 (second change point CP2) (step 58). Then, the level change/return determination unit 354 records, in the memory 340, that there is a blood pressure level change with respect to all the blood pressures of one beat belonging to the third section Z3 (step S8).

On the other hand, it is assumed that the level change/return determination unit 354 determines that the difference between the third average blood pressure level ABL3 and the first average blood pressure level ABL1 is less than the level threshold value ABLth (“NO” in step S7). In this case, the level change/return determination unit 354 proceeds to step S9.

In this example, it is assumed that the difference between the third average blood pressure level ABL3 and the first average blood pressure level ABL1 is less than the level threshold value ABLth. Therefore, the process proceeds to step S9.

In step S9, the level change/return determination unit 354 acquires a period from the tail of the immediately preceding section having no level change to the head of the target section. In this example, the period from the tail of the immediately preceding section Zi having no level change (see first change point CP1) to the head of the target section Z3 (see second change point CP2) is a period T2 (see FIG. 8). Therefore, in step S9, the level change/return determination unit 354 acquires the period T2 as the period from the tail of the immediately preceding section Z1 having no level change to the head of the target section Z3.

Next, in step S10, the level change/return determination unit 354 determines whether or not the period T2 acquired in step S9 is larger than a period threshold value (Tth). Here, for example, it is assumed that the level change/return determination unit 354 determines that the period T2 acquired in step S9 is equal to or less than the period threshold value Tth (“NO” in step S10). In this case, the level change/return determination unit 354 determines that there is no blood pressure level change in the target section Z3 (second change point CP2) (step S70). That is, the level change/return determination unit 354 determines that the blood pressure level in the target section (third section) Z3 has returned to the blood pressure level in the first section. Then, the level change/return determination unit 354 records, in the memory 340, that there is no blood pressure level change (that the blood pressure has returned to a state where there is no level change) with respect to all the blood pressures of one beat belonging to the target section Z3 (step S11).

On the other hand, it is assumed that the level change/return determination unit 354 determines that the period T2 acquired in step S9 is larger than the period threshold value Tth (“YES” in step S10). In this case, the level change/return determination unit 354 makes a blood pressure level change state in the target section Z3 the same as a blood pressure level change state in the section Z2 (referred to as a preceding section) existing between the immediately preceding section Z1 having no level change and the target section Z3 (step S12). Therefore, the level change/return determination unit 354 records, in the memory 340, the same blood pressure level change state as the blood pressure level change state in the second section Z2 that is the preceding section with respect to all the blood pressures of one beat belonging to the target section Z3 (step S12). Here, as described above, it is assumed that there is a blood pressure level change in the second section Z2. Therefore, in step S12, the level change/return determination unit 354 records, in the memory 340, that there is a blood pressure level change with respect to all the blood pressures of one beat belonging to the target section Z3. This is because there is an idea that even if the blood pressure level itself returns, it should not be treated as returning to the normal state if a very long period has elapsed from the tail of the immediately preceding section Z1 having no level change.

Steps S5 to S12 illustrated in FIG. 6 are performed for each section set for the time-series data BTD1 of the maximum blood pressure. Here, in a case where ∞ is adopted as the period threshold value Tth referred to in step S10, step S71 is not substantially performed, and step S70 is always performed.

### (3) Operation of Hospital Terminal 400

In this example, the data indicating a section having a blood pressure level change (for example, the second zone Z2) and a section having no blood pressure level change (for example, first section Z1 and third section Z3) recorded in the memory 340 is transmitted together with the time-series data of blood pressure, as output data, from the blood pressure level change detection apparatus 300 to the hospital terminal 400 via the communication network 50. In this case, the time-series data of blood pressure and the section having a blood pressure level change and the section having no blood pressure level change in the time-series data of blood pressure are displayed on the screen of the display device 420 of the hospital terminal 400.

Therefore, the doctor or the like can grasp the section having a blood pressure level change (for example, the second zone Z2) and the section having no blood pressure level change (for example, first section Z1 and third section Z3) in the time-series data of blood pressure by viewing the screen of the display device 420 of the hospital terminal 400.

The same display as described above can also be performed by the display device 320 of the blood pressure level change detection apparatus 300.

In addition, the time-series data of blood pressure and the time-series data of blood pressure indicating the section having a blood pressure level change and the section having no blood pressure level change in the time-series data of blood pressure may be displayed not only on the screen of the display device 320, 420 but also on a paper surface by, for example, a printer.

### (Effects)

In the blood pressure level change detection apparatus 300 according to the present embodiment, the change point detection unit 352 detects the first change point CP1 in the time-series data of blood pressure (for example, the time-series data BTD1 of the maximum blood pressure). Then, the level change/return determination unit 354 acquires the first average pressure level ABL1 and the second average pressure level ABL2 before and after the first change point CP1, and determines that a blood pressure level change has occurred at the first change point CP1 when a difference between the first average blood pressure level ABL1 and the second average blood pressure level ABL2 is equal to or larger than a predetermined level threshold value ABLth.

Therefore, the blood pressure level change detection apparatus 300 can detect the blood pressure level change in the time-series data BTD1 of blood pressure. Therefore, since a doctor or the like does not need to detect the blood pressure level change from the time-series data BTDI of blood pressure by himself/herself, it is possible to save labor and time for analyzing the time-series data of blood pressure.

In the blood pressure level change detection apparatus 300 of the present embodiment, the change point detection unit 352 detects the second change point CP2 after the first change point CP1 as the change point. The blood pressure level change detection apparatus 300 further includes a section determination unit 353 that divides the time-series data BTD1 of the blood pressure into continuous first section Z1, second section Z2, and third section Z3 by the first change point CP1 and the second change point CP2.

When the blood pressure level change detection apparatus 300 detects the plurality of change points CP1 and CP2, the time-series data BTD1 of blood pressure can be divided into the plurality of sections Z1, Z2, and Z3 by the change points CP1 and CP2. Therefore, the sections Z1, Z2, and Z3 are easily analyzed.

In the blood pressure level change detection apparatus 300 of the present embodiment, when the blood pressure level change has occurred at the first change point CP1, the level change/return determination unit 354 acquires the third average blood pressure level ABL3 by averaging the blood pressure values in a period of a continuous predetermined length immediately after the second change point CP2 for the time-series data BTD1 of blood pressure. Then, when the difference between the third average blood pressure level ABL3 and the first average blood pressure level ABL1 is less than the level threshold value ABLth, the level change/return determination unit 354 determines that the blood pressure level in the third section Z3 has returned to the blood pressure level in the first section Z1.

As described above, the blood pressure level change detection apparatus 300 can determine whether or not the blood pressure level in the third section Z3 has returned to the blood pressure level in the first section Z1. Therefore, it is also possible to determine whether or not to use the blood pressure data included in the third section Z3 as a subsequent analysis target.

In the blood pressure level change detection apparatus 300 of the present embodiment, the first section Z1 is a period of the time-series data BTD1 of blood pressure from the blood pressure measurement start time point to the first change point CP1 detected first after the measurement start.

Therefore, in the blood pressure level change, the first section Z1 can be used as a reference. When a blood pressure level change has occurred in the second section Z2 with respect to the first section Z1, the blood pressure level change detection apparatus 300 can determine whether the blood pressure level in the third section Z3 has returned to the blood pressure level in the first section Z1 as a reference.

### Second Embodiment

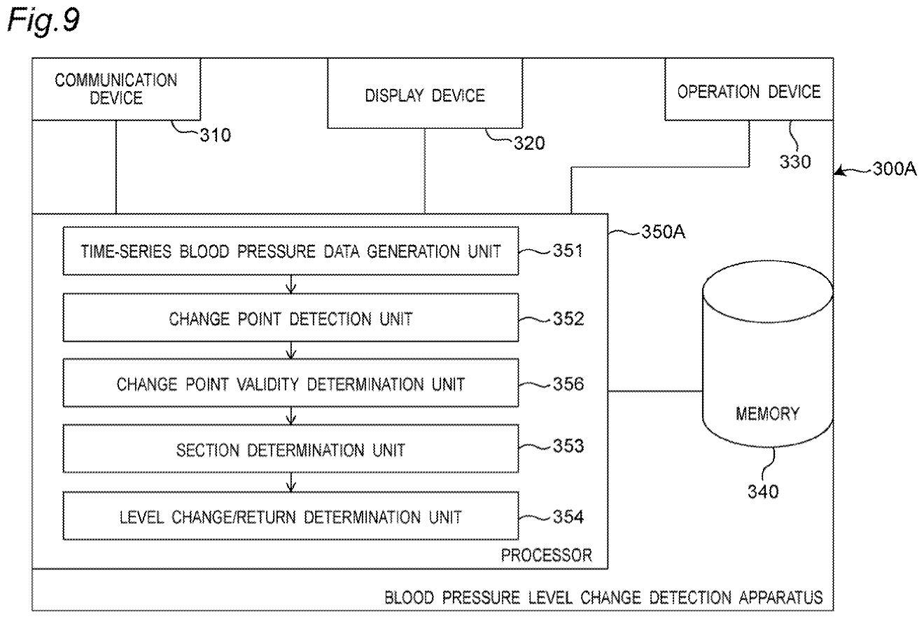

In the first embodiment, the operation of detecting the change points CP1 and CP2 with respect to the time-series data BTD1 of the maximum blood pressure has been described (step S3 in FIG. 6). In the present embodiment, validity of the change points CP1 and CP2 is determined. FIG. 9 illustrates a schematic configuration of a blood pressure level change detection apparatus 300A according to the present embodiment. As can be seen from the comparison between FIGS. 5 and 9, the processor 350A included in the blood pressure level change detection apparatus 300A according to the present embodiment further includes a change point validity determination unit 356 as a functional block. Other configurations are the same as those in the first embodiment.

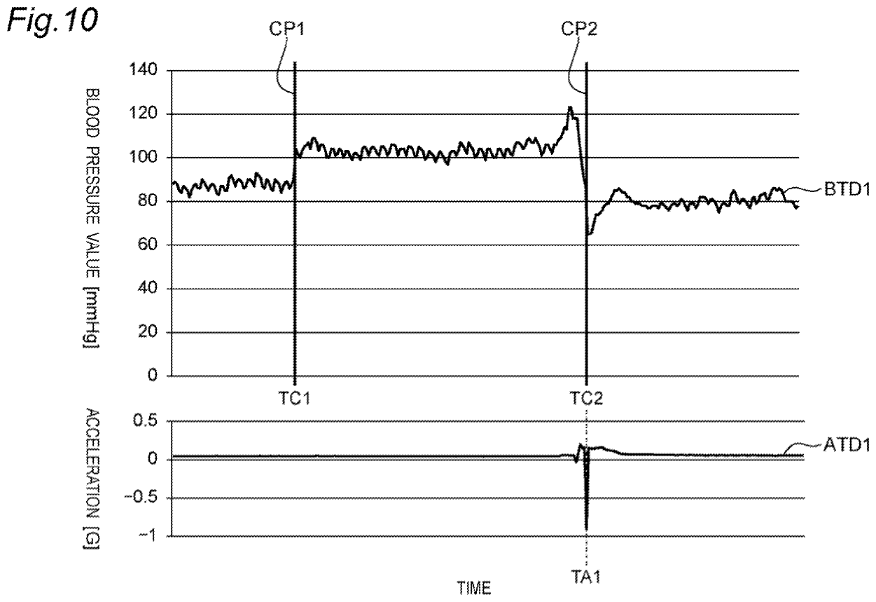

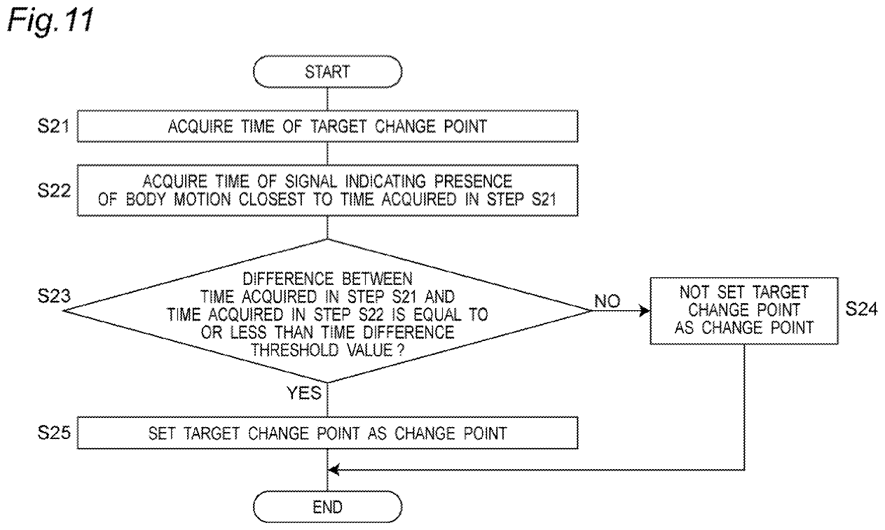

The change point validity determination unit 356 determines the validity of the change points CP1 and CP2 using a body motion signal indicating the body motion of the subject whose blood pressure is to be measured. Here, the sphygmomanometer 200 is attached to the subject. Therefore, the measurement result of the motion sensor 220 of the sphygmomanometer 200 can be adopted as the body motion signal. As an example, the motion sensor 220 is a three-axis acceleration sensor. Next, the operation of determining the validity of the change points CP1 and CP2 will be described with reference to FIGS. 10 and 11.

Here, in FIG. 10, the blood pressure value (mmHg) and the acceleration (G=9.8 m/s²) are adopted as a vertical axis, and time is adopted as a horizontal axis. In the example of FIG. 10, the time-series data BTD1 of the maximum blood pressure and the time-series data ATD1 of acceleration illustrated in FIG. 8 are illustrated. Here, the time-series data BTD1 of the maximum blood pressure and the time-series data ATD1 of acceleration are arranged in the upper part and the lower part of FIG. 10 so that the time axes coincide with each other. In addition, the time-series data ATD1 of the acceleration is data indicating a temporal change of the measurement result by the motion sensor 220. In the present embodiment, since motion sensor 220 is a three-axis acceleration sensor, acceleration in three directions is measured. However, in FIG. 10, only the acceleration value in the y direction is illustrated as the time-series data ATD1 of acceleration for simplification of the drawing. The time-series data BTD1 of the maximum blood pressure is generated through steps S1 and S2 in FIG. 6.

The blood pressure level change detection apparatus 300A receives the measurement data transmitted from the sphygmomanometer 200, and the memory 340 in the blood pressure level change detection apparatus 300A stores the measurement data. Here, the measurement data includes data (motion data) measured by the motion sensor 220 of the sphygmomanometer 200 in addition to the blood pressure value measured by the blood pressure device 210 of the sphygmomanometer 200. The change point validity determination unit 356 reads the motion data from the memory 340. Each motion data is associated with a measurement time. The change point validity determination unit 356 arranges each piece of motion data in time series to generate time-series data ATD1 of acceleration.

FIG. 11 illustrates a flow of determining the validity of the change points CP1 and CP2. Here, FIG. 11 can be understood as a more specific flow of step S3 of FIG. 6 in the case of determining the validity of the change points CP1 and CP2. Hereinafter, the operation after the change points CP1 and CP2 are detected in step S3 of FIG. 6 will be described with reference to FIG. 11.

As described above, in step S3 of FIG. 6, the change point detection unit 352 detects the first change point CP1 and the second change point CP2 for the time-series data BTD1 of the maximum blood pressure (see FIG. 10). After generating the time-series data ATD1 of acceleration, the change point validity determination unit 356 of the processor 350A performs steps S21 to S25 illustrated in FIG. 11 for each of the detected change points CP1 and CP2. Here, the change point that is the target for validity determination is referred to as a target change point.

When the target change point is the first change point CP1, the change point validity determination unit 356 acquires a time TC1 (see FIG. 10) of the target change point CP1 (step S21). Next, the change point validity determination unit 356 acquires the time of the signal indicating the presence of body motion closest to the time TC1 acquired in step S21 (step S22). Here, the time of the signal indicating the presence of body motion is a measurement time point of an acceleration value of a predetermined magnitude or more. In the example of FIG. 10, in the time-series data ATD1 of the acceleration, only one acceleration value equal to or larger than the predetermined magnitude is observed, and the acceleration value is measured at a time TA1. Therefore, in step S22, the change point validity determination unit 356 acquires the time TA1 as the time of the signal indicating the presence of body motion closest to the time TC1.

Next, in step S23, the change point validity determination unit 356 compares a difference between the time TC1 acquired in step S21 and the time TA1 acquired in step S22 with a time difference threshold value (TDth). Here, the time difference threshold value TDth is variably preset in the blood pressure level change detection apparatus 300A. Any value can be adopted as the time difference threshold value TDth. In the following description, the time difference threshold value TDth is in a range of 0 to 1 second in this example.

Specifically, in step S23, the change point validity determination unit 356 determines whether or not the difference between the time TC1 and the time TA1 is equal to or less than the time difference threshold value TDth. In the example of FIG. 10, it is clear that the difference between the time TC1 and the time TA1 is larger than the time difference threshold value TDth (=0 to 1 second). Therefore, the change point validity determination unit 356 determines that the difference between the time TC1 and the time TA1 is larger than the time difference threshold value TDth (“NO” in step S23), and determines that the target change point CP1 is not a valid change point (step S24). That is, the change point validity determination unit 356 determines not to set the target change point CP1 as a change point (step S24). Thereafter, the change point validity determination unit 356 ends the validity determination processing regarding the target change point CP1. Then, the change point validity determination unit 356 changes the target change point to the second change point CP2 and restarts the processing of step S21 and the subsequent steps in FIG. 11.

When the target change point is the second change point CP2, the change point validity determination unit 356 acquires a time TC2 (see FIG. 10) of the target change point CP2 (step S21). Next, the change point validity determination unit 356 acquires the time of the signal indicating the presence of body motion closest to the time TC2 acquired in step S21 (step S22). In the example of FIG. 10, in the time-series data ATD1 of the acceleration, only one acceleration value equal to or larger than the predetermined magnitude is observed, and the acceleration value is measured at a time TA1. Therefore, in step S22, the change point validity determination unit 356 acquires the time TA1 as the time of the signal indicating the presence of body motion closest to the time TC2. Here, it is assumed that time TC2 and time TA1 are the same time.

Next, the change point validity determination unit 356 compares a difference between the time TC2 acquired in step S21 and the time TA1 acquired in step S22 with the time difference threshold value TDth (step S23). Here, as described above, the time difference threshold value TDth is set to 0 to 1 second.

In step S23, the change point validity determination unit 356 determines whether or not the difference between the time TC2 and the time TA1 is equal to or less than the time difference threshold value TDth. As described above, since the time TC2 and the time TA1 are the same time, the difference between the time TC2 and the time TA1 is 0 (zero). Therefore, the change point validity determination unit 356 determines that the difference between the time TC2 and the time TA1 is equal to or less than the time difference threshold value TDth (“YES” in step S23). Then, the change point validity determination unit 356 determines that the target change point CP2 is a valid change point (step S25). That is, the change point validity determination unit 356 determines to adopt the target change point CP2 as the change point (step S25).

For all the change points CP1 and CP2 detected in step S3 of FIG. 6, the change point validity determination unit 356 performs steps S21 to S25 of FIG. 11. Thereafter, the change point validity determination unit 356 performs the section determination processing of step S4 in FIG. 6 by using the second change point CP2 determined to be valid.

### (Effects)

The blood pressure level change detection apparatus 300A of the present embodiment further includes the change point validity determination unit 356. The change point validity determination unit 356 determines the validity of the change points CP1 and CP2 detected by the change point detection unit 352 by using the body motion signal indicating body motion of the subject to be measured for blood pressure.

With the blood pressure level change detection apparatus 300A of the present embodiment, the change points CP1 and CP2 detected by the change point detection unit 352 can be reviewed, and detection points determined to be invalid can also be excluded.

In each of the above embodiments, the processor 260, 350 includes a CPU, but the present invention is not limited thereto. The processor 260, 350 may include a logic circuit (integrated circuit) such as a programmable logic device (PLD) or a field programmable gate array (FPGA).

In each of the embodiments described above, the sphygmomanometer 400 is a tonometry-type sphygmomanometer, but the sphygmomanometer is not limited thereto. The sphygmomanometer 400 may include a light emitting element that emits light toward an artery passing through a corresponding portion of the measurement target site and a light receiving element that receives reflected light (or transmitted light) of the light, and may continuously detect a pulse wave of the artery based on a change in volume (photoelectric type). In addition, the sphygmomanometer 400 may include a piezoelectric sensor in contact with the measurement target site, detect distortion due to pressure of an artery passing through a corresponding portion of the measurement target site as a change in electric resistance, and continuously detect blood pressure based on the change in electric resistance (piezoelectric type). Furthermore, the sphygmomanometer 400 may include a transmission element that transmits a radio wave (transmission wave) toward an artery passing through a corresponding portion of the measurement target site and a reception element that receives a reflected wave of the radio wave, detect a change in distance between the artery and the sensor due to a pulse wave of the artery as a phase shift between the transmission wave and the reflected wave, and continuously detect the blood pressure based on the phase shift (radio wave irradiation method). In addition, as long as the physical quantity with which the blood pressure can be calculated can be observed, other methods may be applied.

The above embodiment is an example, and various modifications can be made without departing from the scope of the present invention. Each of the plurality of embodiments described above can be established independently, but the embodiments can be combined. In addition, various features in different embodiments can be established independently, but features in different embodiments can be combined.

A blood pressure level change detection apparatus according to an embodiment is:

a blood pressure level change detection apparatus for detecting a blood pressure level change in time-series data of blood pressure, the apparatus including:

a change point detection unit that detects a first change point as a change point representing a time at which a blood pressure value has changed beyond a predetermined change rate in the time-series data of blood pressure; and

a level change determination unit that acquires a first average blood pressure level by averaging blood pressure values in a period of a continuous predetermined length immediately before the first change point and acquires a second average blood pressure level by averaging blood pressure values in a period of a continuous predetermined length immediately after the first change point for the time-series data of blood pressure, and determines that a blood pressure level change has occurred at the first change point when a difference between the first average blood pressure level and the second average blood pressure level is equal to or greater than a predetermined level threshold value.

The “first average blood pressure level” is typically regarded as a blood pressure level at the start of measurement (normal time).

In the blood pressure level change detection apparatus of this embodiment, the change point detection unit detects the first change point in the time-series data of blood pressure. Then, the level change determination unit acquires the first average pressure level and the second average pressure level before and after the first change point, and determines that a blood pressure level change has occurred at the change point when a difference between the first average blood pressure level and the second average blood pressure level is greater than or equal to a predetermined level threshold value. Therefore, the blood pressure level change detection apparatus can detect the blood pressure level change in the time-series data of blood pressure. Therefore, since a doctor or the like does not need to detect the blood pressure level change from the time-series data of blood pressure by himself/herself, it is possible to save labor and time for analyzing the time-series data of blood pressure.

In the blood pressure level change detection apparatus of one embodiment,

the change point detection unit detects, as the change point, a second change point after the first change point, and

the blood pressure level change detection apparatus further comprises a section determination unit that divides the time-series data of blood pressure into a first section, a second section, and a third section that are continuous by the first change point and the second change point.

When a plurality of change points is detected by the blood pressure level change detection apparatus of this embodiment, the time--series data of blood pressure can be divided into a plurality of sections by the change points. Therefore, for example, the level change determination can be performed also for the third section.

In the blood pressure level change detection apparatus of one embodiment,

there is a condition that it has been determined by the level change determination unit that the blood pressure level change has occurred at the first change point, and

the blood pressure level change detection apparatus further comprises a level return determination unit that acquires a third average blood pressure level by averaging blood pressure values in a period of a continuous predetermined length immediately after the second change point between the second section and the third section for the time-series data of blood pressure when the condition is satisfied, and determines that the blood pressure level in the third section has returned to the blood pressure level in the first section when a difference between the third average blood pressure level and the first average blood pressure level is less than the level threshold value.

With the blood pressure level change detection apparatus of this embodiment, it is possible to determine whether the blood pressure level in the third section has returned to the blood pressure level in the first section. Therefore, it is also possible to determine whether or not to use the blood pressure data included in the third section as a subsequent analysis target.

In the blood pressure level change detection apparatus of one embodiment,

the first section is a period of the time-series data of blood pressure from a measurement start time point of the blood pressure to the first change point detected first after the measurement start.

According to the blood pressure level change detection apparatus of this embodiment, the first section can be used as a reference in the blood pressure level change. When a blood pressure level change has occurred in the second section with respect to the first section, the blood pressure level change detection apparatus can determine whether the blood pressure level in the third section has returned to the blood pressure level in the first section as a reference.

The blood pressure level change detection apparatus of one embodiment further includes:

a change point validity determination unit that determines validity of the change point detected by the change point detection unit using a body motion signal indicating body motion of a subject whose blood pressure is to be measured.

With the blood pressure level change detection apparatus of this embodiment, the change point detected by the change point detection unit can be reviewed, and the change point determined to be invalid can be excluded.

In another aspect, a blood pressure level change detection method according to the present disclosure is:

a blood pressure level change detection method for detecting a blood pressure level change in time-series data of blood pressure, the method including:

detecting a first change point as a change point representing a time at which a blood pressure value has changed beyond a predetermined change rate in the time-series data of blood pressure;

acquiring a first average blood pressure level by averaging blood pressure values in a period of a continuous predetermined length immediately before the first change point and acquiring a second average blood pressure level by averaging blood pressure values in a period of a continuous predetermined length immediately after the first change point for the time-series data of blood pressure; and

determining that a blood pressure level change has occurred at the first change point when a difference between the first average blood pressure level and the second average blood pressure level is equal to or greater than a predetermined level threshold value.

In the blood pressure level change detection method of the present disclosure, a first change point is detected in time-series data of blood pressure. Then, before and after the first change point, the first average pressure level and the second average pressure level are acquired, and when a difference between the first average blood pressure level and the second average blood pressure level is greater than or equal to a predetermined level threshold value, it is determined that a blood pressure level change has occurred at the first change point. Therefore, for example, by executing the blood pressure level change detection method by a predetermined device, it is possible to detect the blood pressure level change in the time-series data of blood pressure. Therefore, since a doctor or the like does not need to detect the blood pressure level change from the time-series data of blood pressure by himself/herself, it is possible to save labor and time for analyzing the time-series data of blood pressure.

In the blood pressure level change detection method according to one embodiment,

a second change point after the first change point is detected as the change point, and

the time-series data of blood pressure is divided into a first section, a second section, and a third section that are continuous by the first change point and the second change point.

When a plurality of change points is detected by the blood pressure level change detection method of this embodiment, the time-series data of blood pressure can be divided into a plurality of sections by the change points. Therefore, the sections are easily analyzed.

In the blood pressure level change detection method according to one embodiment,

there is a condition that it has been determined that the blood pressure level change has occurred at the first change point,

a third average blood pressure level is acquired by averaging blood pressure values in a period of a continuous predetermined length immediately after the second change point between the second section and the third section for the time-series data of blood pressure when the condition is satisfied, and

it is determined that the blood pressure level in the third section has returned to the blood pressure level in the first section when a difference between the third average blood pressure level and the first average blood pressure level is less than the level threshold value.

With the blood pressure level change detection method of this embodiment, it is possible to determine whether the blood pressure level in the third section has returned to the blood pressure level in the first section. Therefore, it is also possible to determine whether or not to use the blood pressure data included in the third section as a subsequent analysis target.

In the blood pressure level change detection method of one embodiment,

the first section is a period of the time-series data of blood pressure from a measurement start time point of the blood pressure to the first change point detected first after the measurement start.

According to the blood pressure level change detection method of this embodiment, the first section can be used as a reference in the blood pressure level change. When a blood pressure level change has occurred in the second section with respect to the first section, the blood pressure level change detection method can determine whether the blood pressure level in the third section has returned to the blood pressure level in the first section as a reference.

In the blood pressure level change detection method according to one embodiment,

validity of the detected point of change is determined using a body motion signal indicating body motion of a subject whose blood pressure is to be measured.

According to the blood pressure level change detection method of this embodiment, the detected change point can be reviewed, and the change point determined to be invalid can be excluded.

In still another aspect, a program of the present disclosure is a program for causing a computer to execute a blood pressure level change detection method.

The blood pressure level change detection method can be performed by causing a computer to execute the program of the disclosure.

### Effects of the Embodiments

As is clear from the above, according to the blood pressure level change detection apparatus and the blood pressure level change detection method of the present disclosure, it is possible to detect the blood pressure level change in the time-series data of blood pressure.

What is claimed is:

1. A blood pressure level change detection apparatus for detecting a blood pressure level change in time-series data of blood pressure, the apparatus comprising:
a change point detection unit that detects a first change point as a change point representing a time at which a blood pressure value has changed beyond a predetermined change rate in the time-series data of blood pressure; and
a level change determination unit that acquires a first average blood pressure level by averaging blood pressure values in a period of a continuous predetermined length immediately before the first change point and acquires a second average blood pressure level by averaging blood pressure values in a period of a continuous predetermined length immediately after the first change point for the time-series data of blood pressure, and determines that a blood pressure level change has occurred at the first change point when a difference between the first average blood pressure level and the second average blood pressure level is equal to or greater than a predetermined level threshold value.

2. The blood pressure level change detection apparatus according to claim 1, wherein
the change point detection unit detects, as the change point, a second change point after the first change point, and
the blood pressure level change detection apparatus further comprises a section determination unit that divides the time-series data of blood pressure into a first section, a second section, and a third section that are continuous by the first change point and the second change point.

3. The blood pressure level change detection apparatus according to claim 2, wherein
there is a condition that it has been determined by the level change determination unit that the blood pressure level change has occurred at the first change point, and
the blood pressure level change detection apparatus further comprises a level return determination unit that acquires a third average blood pressure level by averaging blood pressure values in a period of a continuous predetermined length immediately after the second change point between the second section and the third section for the time-series data of blood pressure when the condition is satisfied, and determines that the blood pressure level in the third section has returned to the blood pressure level in the first section when a difference between the third average blood pressure level and the first average blood pressure level is less than the level threshold value.

4. The blood pressure level change detection apparatus according to claim 2, wherein the first section is a period of the time-series data of blood pressure from a measurement start time point of the blood pressure to the first change point detected first after the measurement start.

5. The blood pressure level change detection apparatus according to claim 1, which further comprises:
a change point validity determination unit that determines validity of the change point detected by the change point detection unit using a body motion signal indicating body motion of a subject whose blood pressure is to be measured.

6. A blood pressure level change detection method for detecting a blood pressure level change in time-series data of blood pressure, the method comprising:
detecting a first change point as a change point representing a time at which a blood pressure value has changed beyond a predetermined change rate in the time-series data of blood pressure;
acquiring a first average blood pressure level by averaging blood pressure values in a period of a continuous predetermined length immediately before the first change point and acquiring a second average blood pressure level by averaging blood pressure values in a period of a continuous predetermined length immediately after the first change point for the time-series data of blood pressure; and
determining that a blood pressure level change has occurred at the first change point when a difference between the first average blood pressure level and the second average blood pressure level is equal to or greater than a predetermined level threshold value.

7. The blood pressure level change detection method according to claim 6, wherein
a second change point after the first change point is detected as the change point, and
the time-series data of blood pressure is divided into a first section, a second section, and a third section that are continuous by the first change point and the second change point.

8. The blood pressure level change detection method according to claim 7, wherein
there is a condition that it has been determined that the blood pressure level change has occurred at the first change point,
a third average blood pressure level is acquired by averaging blood pressure values in a period of a continuous predetermined length immediately after the second change point between the second section and the third section for the time-series data of blood pressure when the condition is satisfied, and
it is determined that the blood pressure level in the third section has returned to the blood pressure level in the first section when a difference between the third average blood pressure level and the first average blood pressure level is less than the level threshold value.

9. The blood pressure level change detection method according to claim 7, wherein the first section is a period of the time-series data of blood pressure from a measurement start time point of the blood pressure to the first change point detected first after the measurement start.

10. The blood pressure level change detection method according to claim 6, wherein validity of the detected change point is determined by using a body motion signal indicating body motion of a subject whose blood pressure is to be measured.
